(19) United States
(12) Patent Application Publication
(10) Pub. No.: US 2023/0302250 A1
(43) Pub. Date: Sep. 28, 2023

(54) RESPIRATORY MACHINE

(71) Applicant: Sodastream Industries Ltd., Kfar Saba (IL)

(72) Inventors: Avi COHEN, Tel Aviv (IL); Avi COHEN, Shoham (IL)

(73) Assignee: Sodastream Industries Ltd., Kfar Saba (IL)

(21) Appl. No.: 18/020,466

(22) PCT Filed: Aug. 10, 2021
(86) PCT No.: PCT/IL2021/050975

§ 371 Date: Feb. 9, 2023

(30) Foreign Application Priority Data
Aug. 11, 2020 (IL) 276649

(51) Int. Cl. A61M 16/16; A61M 16/12; A61M 16/20; A61M 16/00; A61M 16/10

(52) U.S. Cl. CPC A61M 16/16; A61M 16/12; A61M 16/201; A61M 16/022; A61M 16/0003; A61M 16/108

(57) ABSTRACT

A respiratory machine includes a heat chamber comprising an insulating container and a heating element; a first pipe with an inlet for connecting to a source of at least one gas, wherein some of the first pipe forms a heat exchanger inside the insulating container of the heat chamber, such that when the heating element is actuated the heat exchanger is heated and heats said at least one gas when flowing through the heat exchanger; a humidifier for mixing said at least one gas with vapor to obtain a mixture of humidified heated at least one gas; and a second pipe for delivering the mixture to a breathing port for assisting breathing of a patient.

### FIELD OF THE INVENTION

The present invention relates to respiratory assistance. More particularly, the present invention relates to a respiratory machine.

### BACKGROUND OF THE INVENTION

Respiratory problems may prevent proper lung function. Respiratory disorders include, for example, chronic obstructive pulmonary disease (COPD) illnesses, such as bronchitis, asthma, and emphysema, hereditary diseases like cystic fibrosis (CF), sleep-related disorders like sleep apnea, cancers like lung-cancer and mesothelioma, and infections like tuberculosis, pneumonia, and recently, COVID-19 caused by the new Corona virus.

Patients suffering from breathing problems may require oxygen enriched air to support their breathing to support their lung operation.

There are various known respiratory machines, that include, for example, a ventilator, that provides mechanical ventilation by moving breathable air into and out of the lungs of a patient who is unable to breach independently or has severe breathing difficulties, an oxygen concentrator, that provides oxygen-enriched air, to an inhalator that provides a mixture of oxygen and air for breathing, and is sometimes also used to introduce medicine in the form of vapor for breathing.

### SUMMARY OF THE INVENTION

There is thus provided, in accordance with an embodiment of the invention, a respiratory machine that includes a heat chamber comprising an insulating container and a heating element. The machine also includes a first pipe with an inlet for connecting to a source of at least one gas, wherein some of the first pipe forms a heat exchanger inside the insulating container of the heat chamber, such that when the heating element is actuated the heat exchanger is heated and heats said at least one gas when flowing through the heat exchanger. The machine also includes a humidifier for mixing said at least one gas with vapor to obtain a mixture of humidified heated at least one gas. The machine also includes a second pipe for delivering the mixture to a breathing port for assisting breathing of a patient.

According to some embodiments of the invention, the heat exchanger is designed to be immersed in liquid within the insulating container.

According to some embodiments of the invention, the heating chamber includes a liquid level sensor for detecting changes in a liquid level of the liquid within the insulating container, maintaining free space over the liquid level of the liquid, to facilitate generation of vapor of the liquid in the free space when the liquid is heated.

According to some embodiments of the invention, the first pipe extends out of the heat chamber and is connected to a Venturi eductor, the Venturi eductor connected via an auxiliary pipe to the free space for suction of vapor into the Venturi eductor to mix with said at least one gas.

According to some embodiments of the invention, the first pipe is connected to a diffuser for diffusing said at least one gas with the vapor inside the free space, and wherein the insulating container includes an outlet for connecting to the second pipe.

According to some embodiments of the invention, the heat exchanger is embedded in a heat conducting block inside the heat chamber.

According to some embodiments of the invention, free space is provided over the heat conducting block inside the insulating container to facilitate generation of vapor in the free space when liquid is introduced into the free space.

According to some embodiments of the invention, the first pipe includes a three way valve for connecting to two gas ports.

According to some embodiments of the invention, the three way valve is configured to allow adjusting a desired mixing ratio of gases from the two gas ports.

According to some embodiments of the invention, the two gas ports comprise an air port and an oxygen port.

According to some embodiments of the invention, the respiratory machine further includes a controller for monitoring and controlling operation of the machine.

According to some embodiments of the invention, the controller is configured to receive sensed data from a plurality of sensors monitoring operation parameters of the respiratory machine and vital signs parameters of the patient, and to adjust operation of the respiratory machine according to a predetermined algorithm.

According to some embodiments of the invention, the controller is configured to adjust one or more work parameters of the respiratory machine selected from the group of parameters consisting of: gas mixture of said at least one gas, flow rate of said at least one gas through the second pipe and temperature of said at least one gas passing through the heat exchanger.

According to some embodiments of the invention, the insulating container is disposable.

According to some embodiments of the invention, the second pipe is disposable.

According to some embodiments of the invention, a respiratory machine is provided, that includes a heat chamber comprising an insulating container and a heating element; a plurality off first pipes, each with an inlet for connecting to a source of at least one gas, wherein some of each of the first pipes forms a heat exchanger inside the insulating container of the heat chamber, such that when the heating element is actuated the heat exchanger is heated and heats said at least one gas when flowing through the heat exchanger; a humidifier for mixing said at least one gas with vapor to obtain a mixture of humidified heated at least one gas; and a plurality of second pipes, each for delivering the mixture to a breathing port for assisting breathing of a patient.

### BRIEF DESCRIPTION OF THE DRAWINGS

In order for the present invention to be better understood and for its practical applications to be appreciated, the following Figures are provided and referenced hereafter. It should be noted that the Figures are given as examples only and in no way limit the scope of the invention. Like components are denoted by like reference numerals.

FIG. 1 is a schematic illustration of a respiratory machine, according to some embodiments of the present invention.

FIG. 2 is a schematic illustration of another respiratory machine, according to some embodiments of the present invention.

FIG. 3 is a schematic illustration of another respiratory machine, according to some embodiments of the present invention.

FIG. 4 is a schematic illustration of a combined conductive heater and humidifier for incorporation in a respiratory machine, according to some embodiments of the present invention.

FIG. 5 is a schematic illustration of a disposable heating chamber, for incorporation in a respiratory machine, according to some embodiments of the present invention.

FIG. 6 is a schematic illustration of a respiratory machine for use by a plurality of patients, according to some embodiments of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options).

The recent outbreak of the COVID-19 pandemic has been characterized by increased numbers of patients who need breathing assistance, and increased numbers of patients in critical condition who are anesthetized and ventilated. Patients suffering from breathing problems (e.g., patients having COVID-19) can greatly benefit from a steady supply of oxygen-enriched humid air that is heated to human body temperatures, that enhances their breathing and improves oxygen intake.

Essentially, a respiratory machine, according to some embodiments of the invention may be designed to provide a patient (or patients), e.g., through a breathing port such as a nasal cannula or a face mask, oxygen-enriched air, or other gas or mixture of gases, at a substantially constant temperature, e.g., within or near human body (or animal body) temperature range (for example, 35-38 degrees centigrade), and at a high level of humidity to assist the patient's breathing.

A respiratory machine according to some embodiments of the present invention is aimed at reducing the patient's breathing effort, provide humidified warmed air (or other gas), using humidified warmed gas to retain mucus fluidity, which is known to be beneficial in the recovery of airways.

An aspect of some embodiments of the present invention, is passing oxygen-enriched air, or other gas or mixture of gases (hereinafter it is referred to as “oxygen-enriched air”, or “gas” for brevity, without derogating generality), through a pipe, which is thermally coupled to a heat exchanger, or that forms a heat exchanger, so as to heat the enriched air to a desired predetermined temperature. Another aspect of some embodiments of the present invention is mixing the heated enriched air with vapor (e.g., water vapor) which is maintained at a high level of humidity (e.g., over 70%, over 80%, over 90%, etc.), to obtain humidified oxygen enriched air. Another aspect of some embodiments of the present invention is maintaining a flow of the humidified oxygen enriched air at a predetermined substantially constant flow rate (e.g., a constant flow rate in the range of 40-70 liters per minute—LPM, in the range of 60-100 LPM, etc.). Another aspect of some embodiments of the present invention is the provision of a respiratory machine that may be used by a single patient. Another aspect of some embodiments of the present invention is the provision of a respiratory machine that may be used by a plurality of patients.

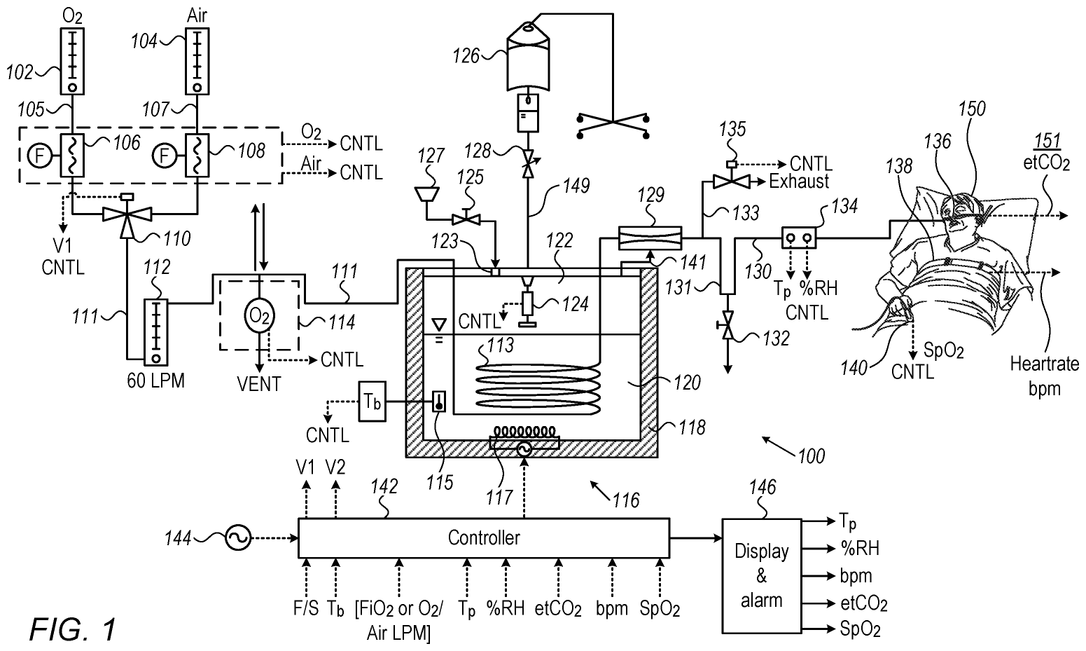

FIG. 1 is a schematic illustration of a respiratory machine 100, according to some embodiments of the present invention.

A respiratory machine, according to some embodiments of the present invention may be operated by a human user (e.g., medical staff personnel, hereinafter—“user”) and/or automatically controlled and operated by a controller 142. For the sake of brevity, instead of adding to the figures long connecting lines between various controllable components and controller 142, the controllable components are marked in the figures with “CNTL” to indicate link to the controller 142. Such link may be a wired link or a wireless link.

Respiratory machine may be linked to oxygen supply port 102 and air supply port 104 via pipes 105 and 107, respectively equipped with flow rate sensors 106 and 108, respectively.

A three-way valve 110 may be provided, intercepting pipes 105 and 207 to facilitate adjusting of the oxygen-air ratio. The adjustment of the oxygen-air ratio may be carried out manually by a user or by controller 142, based on sensed data by the flow rate sensors 106 and 108. The outlet of three-way valve 110 may be connected to an inlet of pipe 111 (the outlet and inlet may be separable or permanently linked).

Flow meter 112 may be provided to measure the flow rate of the mixed gas (e.g., 60 LPM) within pipe 111.

In some embodiments of the present invention, instead of monitoring and controlling flow rate sensors 106 and 108 (e.g., by controller 142), a FiO2 (fraction of inspired Oxygen) sensor 114 may be placed along pipe 111, and used for monitoring the mix ratio between oxygen and air.

Pipe 111 may extend into heating chamber 116 that comprises closed insulated container 118 made from insulating material (e.g., polystyrene, Styrofoam, etc.) or insulating structure (double walled with a space in between for heat insulation).

Heating chamber 116 defines a predefined internal insulated space within, that may be partially occupied by water (e.g., tap water, medical water, sterilized water, saline, etc.) or other liquid (e.g., mixture of water and medication), leaving free space 122 above the liquid level, which may fill up with vapor, when liquid 120 is heated up.

Heating element 117 may be provided, for example at the bottom of heating chamber 116, for heating the liquid contained in the heating chamber 116.

A liquid level sensor, e.g., float valve switch 124 may be provided within heating chamber 116 to detect changes in the liquid level and when the liquid level drops below a predetermined minimal liquid level facilitates flow of more liquid via pipe 149 that links a liquid reservoir 126 (e.g., a bag of sterile water) to the inside of heating chamber 116, and when the liquid level surpasses a predetermined maximal level prevents anymore liquid from flowing into the heating chamber 116. Liquid reservoir 126 may include a flow restrictor 128 (e.g., drip adjuster) to control the flow rate of liquid via pipe 149 into heating chamber 116. Fill port 127 may be also provided, controlled by valve 125 for introducing the same or other type of liquid or additive into heating chamber 116 via inlet 123. A temperature sensor115 may be provided for monitoring the temperature of the liquid within heating chamber 116.

Pipe 111, within heat chamber 116, is arranged to form a heat exchanger 113, for example by forming a coiled structure to extend the outside surface of pipe 111 that is in contact with liquid 120. When heating element 117 is actuated liquid 120 may be heated to a desired predetermined temperature, e.g., to a temperature within a body temperature range, such as in the range of 35-38 degrees centigrade, causing heated vapor to accumulate within free space 122.

Pipe 111 eventually emerges from within heating chamber and is linked to a humidifier, e.g., Venturi eductor 129 and the gas that flows within the Venturi eductor serves as the driving fluid that generates suction to suck vapor from free space 122 of the heating chamber 116 through auxiliary pipe 141 that links the space above the liquid level to the Venturi eductor so as to mix the vapor with the gas to obtain heated humidified mixed gas. The heated humidified mixed gas may then flow via pipe 130 emerging from Venturi eductor 129. Pipe 130 may include a knee bend to capture any liquid that is found within pipe 130 (e.g., condensed vapor or gas), which may be evacuated from the pipe via valve 132. Exhaust pipe 133 may be provided, branching out of pipe 130, and may include an exhaust valve 135 for allowing exhausting excess gas from pipe 130, when necessary or desired, e.g., for regulating the flow rate to the patient

Pipe 130 may be provided with a sensing unit with one or more sensors 134 to sense the temperature and/or humidity of the mixed gas within pipe 130. The distal end of pipe 130 is linked to a nasal cannula 136 or a face mask, that may be placed over the nose and/or mouth of the patient 150, through which the heated mixture of gas and vapor is discharged at a predetermined flow rate (e.g., 60 LPM). Other vital parameters of patient 150 may also be monitored. For example, the heart rate of patient 150 may be monitored, e.g., using heartbeat sensing strap 138, oxygen saturation in the blood (SpO₂) may also be monitored using oximeter sensor 140, end-tidal CO₂ may be measured, e.g, by a sensor at the nasal cannula, blood pressure (e.g., using a blood pressure sensor—cuff etc., sensing blood pressure in mmHg).

Pipe 130 and/or pipe 111 may be formed as a single continuous pipe or may be formed of two or more separable segments. Some or all of the separable segments of pipe 130 and/or pipe 111 may be disposable, for use by a single patient.

According to some embodiments of the present invention, various components of the respiratory machine may be controlled and/or operated by controller 142. Controller 142 may include or be linked to a display device 146 for displaying information (e.g., various vital signs parameters, messages and/or alerts.

Controller 142 may be linked to various components of the respiratory machine 100. For example, controller 142 may be linked to any, some or all of the following components: flow rate sensors 106 and 108, three-way valve 110, Fio2 sensor 114, heating element 117, temperature sensor 115, exhaust valve 135, water level sensor/switch 124, sensors 134 that measure the temperature and humidity of the mixed gas in pipe 130, sensors for measuring vital parameters (e.g., heartbeat rate sensor 138, oximeter 140, end-tidal CO₂ sensor 151, etc.). Respiratory machine 100 may be powered by the mains electric supply 144 (e.g., via electric socket providing voltage for electric appliances, such as 110V, 220V etc.).

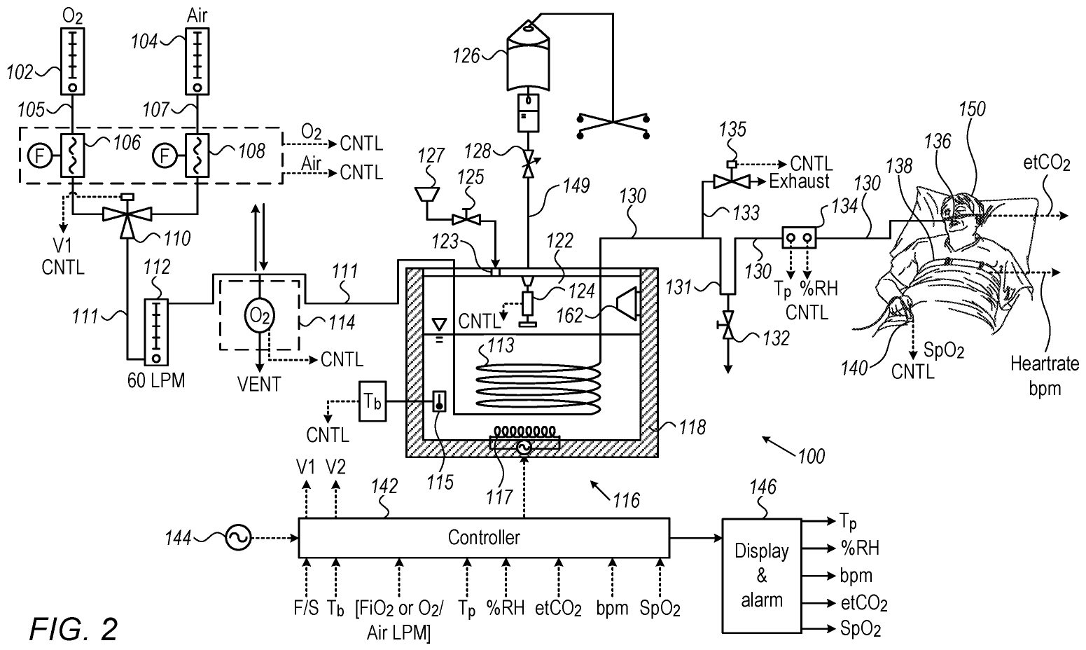

FIG. 2 is a schematic illustration of another respiratory machine 200, according to some embodiments of the present invention.

Most of the design of respiratory machine 200 is quite similar to the design of respiratory machine 100 shown in FIG. 1. However, instead of using a Venturi eductor, the mixing of gas with vapor is achieved by directing the heated gas flowing in tube 111 through a diffuser 162 that is located inside the heating chamber, and discharging the gas into free space 122, diffused in order to maximize collisions between gas particles and vapor particles. The pressure within free space 122 caused by the inflow of gas causes the mixture of gas and vapor to flow out of the heating chamber via tube 130, whose inlet is located at the wall of the insulated container 118 of the heating chamber 116, and fluidically connected with the free space 122, to the nasal cannula 136 of patient 150.

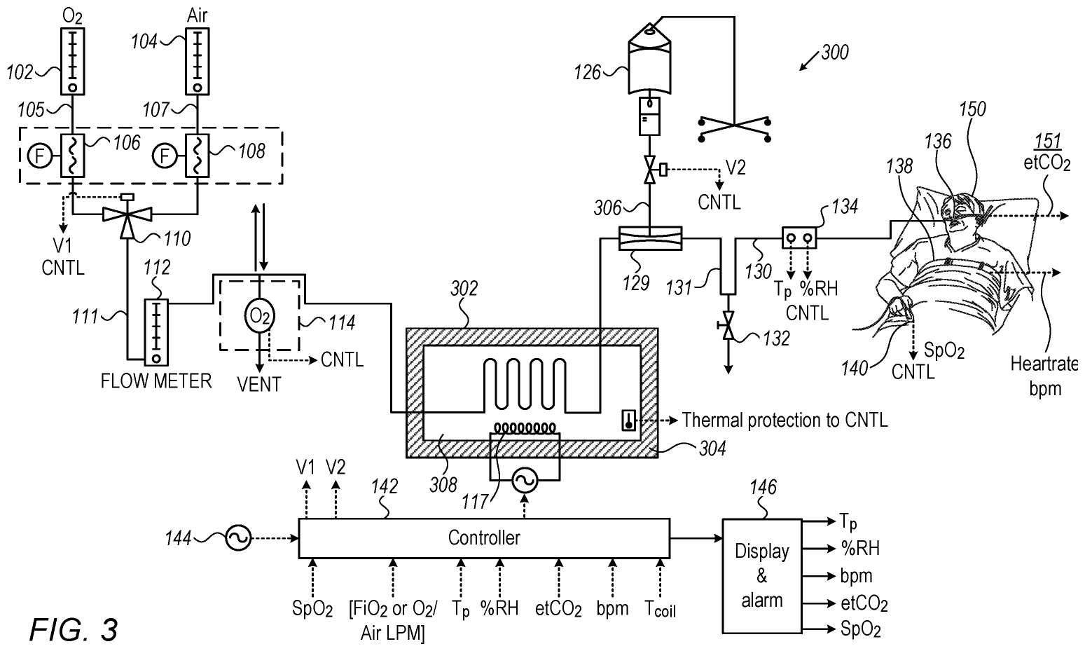

FIG. 3 is a schematic illustration of another respiratory machine, according to some embodiments of the present invention. In the respiratory machine of FIG. 3 the heat exchanger 113 is embedded in a heat chamber 302, which includes a heat conducting block 308, for example, heat conductive metal like copper, aluminum etc. Heat conducting block 308 may be housed in insulating container 304 to prevent undesired heat dissipation and burns inflicted by accidental contact with heat conducting block 308. Gas flowing through heat exchanger 113 is heated and then flows into Venturi eductor 129 where it serves as a driving fluid causing suction of liquid droplets of sterile water or other liquid stored in bag 126, that may flow via valve 128 and via auxiliary pipe 306 into Venturi eductor 129. The humidified and warmed air-oxygen mixture flows through pipe 130 and is then discharged at the nasal cannula 136 of patient 150.

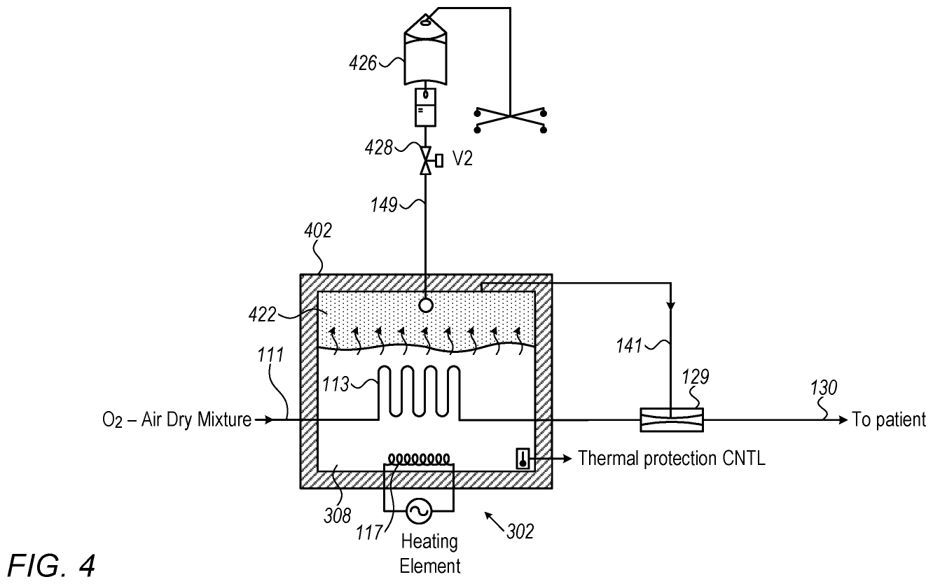

FIG. 4 is a schematic illustration of a combined conductive heater and humidifier for incorporation in a respiratory machine, according to some embodiments of the present invention. In this embodiment heat chamber 302 includes an insulating container 402 in which heat conducting block 308 is placed. Tube 111 is embedded in heat conducting block 308 forming heat exchanger 113 inside the block. Heat conducting block 308 does not fully occupy the inside of insulating container 402, leaving a free space 422. Liquid (e.g., water, sterile water, etc. contained in a liquid reservoir 426 with a flow controller 428) from liquid may drip into free space 422 through pipe 149 that introduces the dripping liquid into free space 422. Heat conducting block 308 heats up the liquid to generate vapor. Gas flows through pipe 111 and is heated in the heat chamber 302. The heated gas flows into Venturi eductor 129 and vapor from free space 422 is sucked into Venturi eductor 129 via auxiliary pipe 141 and mixed with the heated gas. The mixture of gas and vapor flows through pipe 130 to nasal cannula 136 (see FIG. 3).

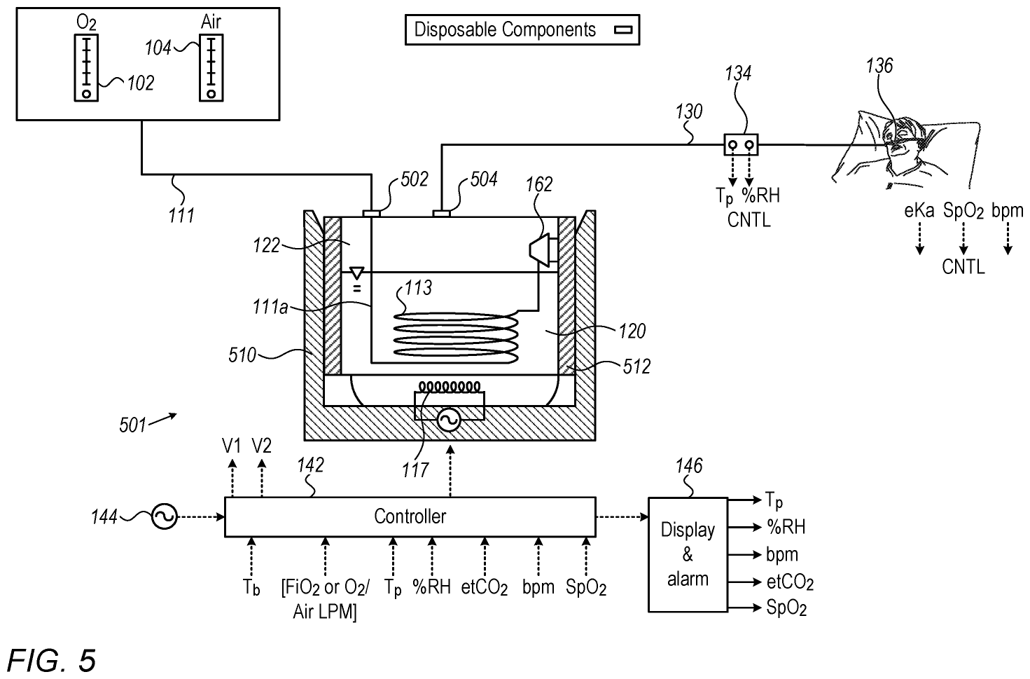

FIG. 5 is a schematic illustration of a disposable heating chamber, for incorporation in a respiratory machine, according to some embodiments of the present invention. Heat chamber 501 may include a permanent base 510. Base 510 may be made of insulating material and include a heating element 117. Base 510 may be designed to contain a disposable insulating container 512 in which pipe 111a extends from an inlet located on the wall of insulating container 512 linked to inlet fitting 502. Pipe 111 for delivering gas (e.g., a mixture of gases from oxygen supply port 102 and air supply port 104) may be connected to pipe 111a via fitting 502. Heating chamber 501 is of the type that includes a gas pipe—111a—immersed in liquid 120 and forming a heat exchanger 113 for heating the gas that flows inside pipe 111a when the liquid is heated by heating element 117. Pipe 111a ends with diffuser 162 that diffuses the gas into free space 122. Outlet fitting 504 is provided to which pipe 130 may be connected, and the mixture of heated gas and vapor generated over the liquid level within free space 122 is discharged through pipe 130 and delivered to nasal cannula 136. The insulating container with its fittings and pipe 130 may be disposable, for use by one patient only. When a new patient requires the respiratory machine, a new disposable insulating container 512 (and with pipe 130) may be installed into base 510 and used by another patient.

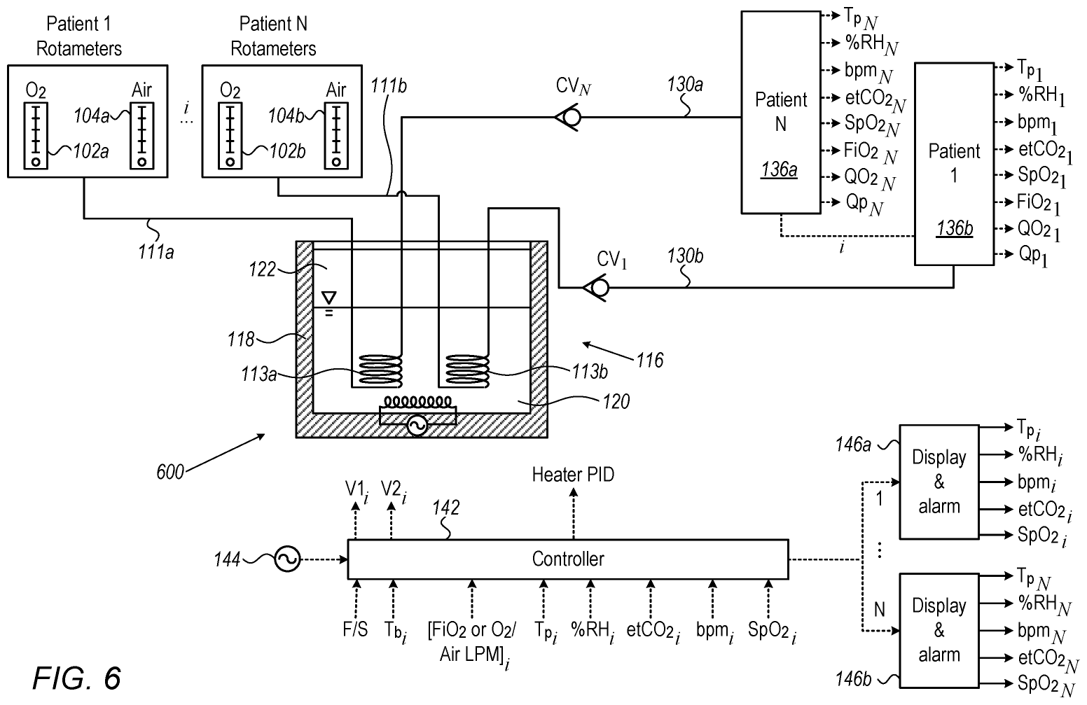

FIG. 6 is a schematic illustration of a respiratory machine 600 for use by a plurality of patients, according to some embodiments of the present invention. Respiratory machine 600 may include a shared heating chamber 116. A plurality of gas supply pipes (e.g., 111a, 111b) are provided for delivering gas, e.g., from oxygen supply ports 102a, 102b and air supply ports 104a, 104b, respectively, and extend into heating chamber 116 to be submerged in liquid 120, forming heat exchangers 113a, 113b respectively, or embedded in heat conducting block, etc. Separate humidifiers (not shown in this figure, for brevity, see FIGS. 1, 2, 3 and 4 for examples of humidifiers) are provided to mix the gas with vapor for each patient and deliver the mixture of heated gas and vapor to the nasal cannulas 136a 136b via pipes 130a 130b respectively. In some embodiments of the invention pipes 130a and 130b may be disposable and replaced when required for use by other patients.

A respiratory machine, according to some embodiments of the present invention can be fully autonomous. The controller may obtain various sensed work parameters relating to the operation of the respiratory machine, e.g., flow rates, FiO2, temperature etc. and also obtain various sensed vital signs data relating to the patient. The controller may be configured to execute a predetermined algorithm that monitors sensed vital signs of the patient (e.g., SpO2, heartbeat rate, etCO2 etc.) and analyzes in real-time the required action and adjustment of the work parameters of the respiratory machine. For example, when the patient sensed vital signs are: etCO2 40 mmHg, SpO2 90%, heartrate 80 bpm, respiratory rate 15 breaths/min, the controller generates adjustment commands to one or more controlled components of the respiratory machine, e.g., three-way valve 110, FiO2 and/or exhaust valve 135 for low rate regulation and/or the heating element 117 to adjust the temperature of the gas passing through the heat exchanger, according to recent accumulative data of the patient and wait a pre-determined time for a change of vital signs, then adjust the machine parameters again and so forth.

Different embodiments are disclosed herein. Features of certain embodiments may be combined with features of other embodiments. Thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A respiratory machine comprising:
a heat chamber comprising an insulating container and a heating element;
a first pipe with an inlet for connecting to a source of at least one gas, wherein some of the first pipe forms a heat exchanger inside the insulating container of the heat chamber, such that when the heating element is actuated the heat exchanger is heated and heats said at least one gas when flowing through the heat exchanger;
a humidifier for mixing said at least one gas with vapor to obtain a mixture of humidified heated at least one gas; and
a second pipe for delivering the mixture to a breathing port for assisting breathing of a patient.

2. The machine of claim 1, wherein the heat exchanger is designed to be immersed in liquid within the insulating container.

3. The machine of claim 2, wherein the heating chamber includes a liquid level sensor for detecting changes in a liquid level of the liquid within the insulating container, maintaining free space over the liquid level of the liquid, to facilitate generation of vapor of the liquid in the free space when the liquid is heated.

4. The machine of claim 3, wherein the first pipe extends out of the heat chamber and is connected to a Venturi eductor, the Venturi eductor connected via an auxiliary pipe to the free space for suction of vapor into the Venturi eductor to mix with said at least one gas.

5. The machine of claim 3, wherein the first pipe is connected to a diffuser for diffusing said at least one gas with the vapor inside the free space, and wherein the insulating container includes an outlet for connecting to the second pipe.

6. The machine of claim 1, wherein the heat exchanger is embedded in a heat conducting block inside the heat chamber.

7. The machine of claim 6, wherein free space is provided over the heat conducting block inside the insulating container to facilitate generation of vapor in the free space when liquid is introduced into the free space.

8. The machine of claim 1, wherein the first pipe includes a three way valve for connecting to two gas ports.

9. The machine of claim 8, wherein the three way valve is configured to allow adjusting a desired mixing ratio of gases from the two gas ports.

10. The machine of claim 8, wherein the two gas ports comprise an air port and an oxygen port.

11. The machine of any of claims 1 to 10, further comprising a controller for monitoring and controlling operation of the machine.

12. The machine of claim 11, wherein the controller is configured to receive sensed data from a plurality of sensors monitoring operation parameters of the respiratory machine and vital signs parameters of the patient, and to adjust operation of the respiratory machine according to a predetermined algorithm.

13. The machine of claim 12, wherein the controller is configured to adjust one or more work parameters of the respiratory machine selected from the group of parameters consisting of: gas mixture of said at least one gas, flow rate of said at least one gas through the second pipe and temperature of said at least one gas passing through the heat exchanger.

14. The machine of claim 1, wherein the insulating container is disposable.

15. The machine of claim 14 wherein the second pipe is disposable.

16. A respiratory machine comprising:
a heat chamber comprising an insulating container and a heating element;
a plurality off first pipes, each with an inlet for connecting to a source of at least one gas, wherein some of each of the first pipes forms a heat exchanger inside the insulating container of the heat chamber, such that when the heating element is actuated the heat exchanger is heated and heats said at least one gas when flowing through the heat exchanger;
a humidifier for mixing said at least one gas with vapor to obtain a mixture of humidified heated at least one gas;
a plurality of second pipes, each for delivering the mixture to a breathing port for assisting breathing of a patient.
